(19) United States
(12) Patent Application Publication
(10) Pub. No.: US 2026/0263143 A1
(43) Pub. Date: Sep. 10, 2026

(54) MOTORIZED VESSEL SEALER OVERRIDE

(71) Applicant: COVIDIEN LP, Mansfield, MA (US)

(72) Inventor: Daniel A. JOSEPH, Lafayette, CO (US)

(73) Assignee: COVIDIEN LP, Mansfield, MA (US)

(21) Appl. No.: 18/881,542

(22) PCT Filed: Jul. 6, 2023
(86) PCT No.: PCT/IB2023/057009

§ 371 Date: Jan. 6, 2025

Related U.S. Application Data
(60) Provisional application No. 63/358,887, filed on Jul. 7, 2022.

(51) Int. Cl. A61B 18/14; A61B 17/00; A61B 18/00; A61B 90/00

(52) U.S. Cl. CPC A61B 18/1445; A61B 90/06; A61B 2017/00398; A61B 2018/00184; A61B 2018/0063; A61B 2018/0091; A61B 2090/064

(57) ABSTRACT

A surgical forceps for sealing tissue includes a housing having a drive rod with an end effector including first and second jaw members configured to seal tissue upon electrical activation thereof. A stationary handle depends from the housing and is configured to support a clamping override actuator operably coupled to a motor. A moveable handle operably couples to the drive rod and is movable relative to the stationary handle through an initial range of motion to move the drive rod to pivot the jaw members relative to one another to grasp tissue therebetween. The moveable handle is configured to contact the clamping override actuator through a further range of motion to actuate the clamping override actuator to engage the motor with the drive rod to further move the drive rod to generate a higher clamping force to seal tissue.

### BACKGROUND

### Technical Field

The present disclosure relates generally to the field of surgical instruments. In particular, the disclosure relates to an in-line, endoscopic electrosurgical forceps that is economical to manufacture and is capable of sealing and cutting tissue structures.

### Background of Related Art

With tissue sealing, surgeons typically have to generate enough clamping force prior to initiating energy delivery which over repeated use can cause fatigue with in-line devices. Moreover, in-line devices typically do not have locking features further requiring the surgeon to hold the forceps in a “clamped” position until the generator recognizes a seal completion.

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To affect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

In-line electrosurgical forceps are one common type of electrosurgical instrument which offers the ease of electrically activating the forceps when fully and continuously compressing the same handle used to close the jaw members about tissue. In some instances, the surgeon may simply desire to grasp tissue and not electrically activate the jaw members. As such, it would be desirous to manufacturer an in-line electrosurgical forceps that facilitates both grasping tissue and in-line activation.

### SUMMARY

Provided in accordance with the present disclosure is a surgical forceps for sealing tissue which includes a housing having an elongated drive rod with an end effector assembly disposed at a distal end thereof, the end effector including first and second jaw members configured to seal tissue upon electrical activation thereof. A stationary handle depends from the housing and is configured to support a clamping override actuator disposed therein, the clamping override actuator is operably coupled to a motor. A moveable handle is operably coupled to the drive rod and is movable relative to the stationary handle through an initial range of motion to move the drive rod upon actuation thereof without contacting the clamping override actuator to pivot one or both of the first or second jaw members relative to the other of the first or second jaw members to grasp tissue therebetween. The moveable handle is configured to contact the clamping override actuator through a further range of motion to actuate the clamping override actuator to engage the motor with the drive rod to further move the drive rod to generate a higher clamping force to seal tissue. The clamping override actuator is disposed in the actuation path of the moveable handle.

In aspects according to the present disclosure, the clamping override actuator includes an encoder configured to communicate to the motor that the clamping override actuator has been contacted by the moveable handle, which, in turn, recognizes that the motor is mechanically engaged with the drive rod.

In aspects according to the present disclosure, the clamping override actuator includes a key operably connected thereto that is movable therewith, the key is configured to urge one or more gears from the motor into engagement with one or more corresponding gears on the drive rod. In other aspects according to the present disclosure, the motor includes a gear box disposed on a drive shaft that is configured to matingly engage a corresponding series of gears disposed on the drive rod.

In aspects according to the present disclosure, the clamping override actuator is biased against a spring disposed within the stationary handle.

In aspects according to the present disclosure, the moveable handle is moveable about a pivot through the initial range of motion to force a drive collar against the drive rod to pivot the jaw members relative to one another for grasping tissue. In other aspects according to the present disclosure, the drive collar compresses a spring against a drive collar stop to regulate the grasping pressure between the jaw members. In still other aspects according to the present disclosure, the forceps further includes a position encoder disposed proximate the drive collar stop configured to report the position of the drive collar stop relative thereto during sealing. In yet other aspects according to the present disclosure, the forceps includes a force transducer disposed on a portion of the drive rod proximate the drive collar stop configured to sense a linear force on the drive rod during sealing.

Provided in accordance with the present disclosure is a surgical forceps for sealing tissue which includes a housing having an elongated drive rod with an end effector assembly disposed at a distal end thereof, the end effector including first and second jaw members configured to seal tissue upon electrical activation thereof. A stationary handle depends from the housing and is configured to support a clamping override actuator disposed therein, the clamping override actuator is operably coupled to a motor. A moveable handle is operably coupled to the drive rod and is movable relative to the stationary handle through an initial range of motion which moves the drive rod upon actuation thereof to pivot one or both of the first or second jaw members relative to the other of the first or second jaw members to grasp tissue therebetween under a first clamping pressure by pivoting a drive collar against a drive stop. The moveable handle is configured to transition through a further range of motion to an override position to actuate a clamping override actuator to engage a motor with the drive rod to further move the drive rod to generate a higher clamping force to seal tissue. The clamping override actuator is disposed in the actuation path of the moveable handle.

In aspects according to the present disclosure, the clamping override actuator includes an encoder configured to communicate to the motor that the clamping override actuator has been contacted by the moveable handle, which, in turn, recognizes that the motor is mechanically engaged with the drive rod.

In aspects according to the present disclosure, the clamping override actuator includes a key operably connected thereto that is movable therewith, the key is configured to urge one or more gears from the motor into engagement with one or more corresponding gears on the drive rod. In other aspects according to the present disclosure, the motor includes a gear box disposed on a drive shaft that is configured to matingly engage a corresponding series of gears disposed on the drive rod.

In aspects according to the present disclosure, the clamping override actuator is biased against a spring disposed within the stationary handle.

In aspects according to the present disclosure, the moveable handle is moveable about a pivot through the initial range of motion to force a drive collar against the drive rod to pivot the jaw members relative to one another for grasping tissue. In other aspects according to the present disclosure, the drive collar compresses a spring against a drive collar stop to regulate the grasping pressure between the jaw members. In still other aspects according to the present disclosure, the forceps further includes a position encoder disposed proximate the drive collar stop configured to report the position of the drive collar stop relative thereto during sealing. In yet other aspects according to the present disclosure, the forceps includes a force transducer disposed on a portion of the drive rod proximate the drive collar stop configured to sense a linear force on the drive rod during sealing.

Provided in accordance with another embodiment of the present disclosure is a method of sealing tissue with motorized assistance and includes and initial step of orienting tissue between first and second jaw members of an end effector assembly supported at a distal end of an elongated drive rod extending from a housing. The method further includes: moving a movable handle through an initial range of motion relative to a stationary handle depending from the housing to move the drive rod to approximate the jaw members to grasp tissue disposed therebetween under a first pressure; moving the handle through a further range of motion to transition to an override position of the moveable handle so as actuate a clamping override actuator which engages a motor with the drive rod, the motor configured to relieve the moveable handle from the drive rod and further move the drive rod to generate higher clamping forces for sealing tissue; and releasing the clamping override actuator upon seal completion to disengage the motor and re-engage the moveable handle with the drive rod to open the jaw members upon movement thereof to release the tissue.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is an internal side view of an in-line, electrosurgical forceps according to an embodiment of the present disclosure including a housing, an elongated shaft, an end effector assembly having opposing jaw members and a movable handle;

FIGS. 2A-2C are various views of the movable handle of the forceps of FIG. 1 and the internal components therein showing the varying range of grasping positions;

FIGS. 2D and 2E are various views of the movable handle of the forceps of FIG. 1 and the internal components therein showing activation of a clamping over-ride through the continuous range of motion of the movable handle activating a motor to provide a higher clamping force onto tissue disposed between the jaw members;

FIG. 2F is an internal side view of the forceps of FIG. 1 showing the movable handle releasing the clamping over-ride and releasing the tissue;

FIG. 3 is an enlarged internal view of another embodiment of an electrosurgical forceps including a motor-driven over-ride according to the present disclosure; and

FIGS. 4A-4D are various internal views showing the movable handle and internal cooperating components of the over-ride to provide a higher clamping force onto tissue disposed between the jaw members when actuated.

### DETAILED DESCRIPTION

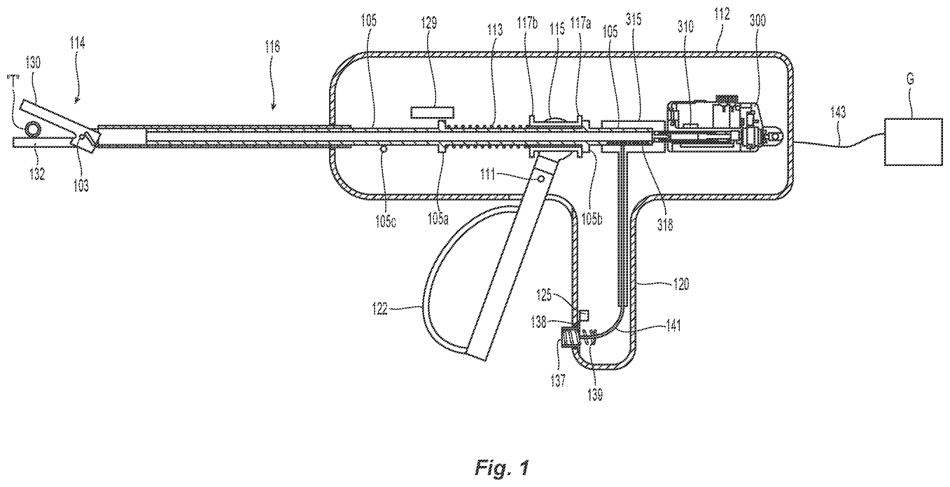

Referring initially to FIG. 1, an electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half and a right housing half as perceived by an operator using the forceps 100 and may be constructed of sturdy plastic which are joined to one another by adhesives, ultrasonic welding or other suitable assembly methods.

To mechanically control the end effector 114, the housing 112 supports a stationary handle 120 and a movable handle 122. Typically, a trigger and a rotation knob are included in the forceps 100 design for reciprocating a knife for cutting tissue disposed between the jaw members and for rotating the jaw members but are not described herein for the purposes of brevity. A detailed discussion of these features may be found in commonly-owned U.S. Pat. No. 7,255,697, the entire contents of which being incorporated by reference herein.

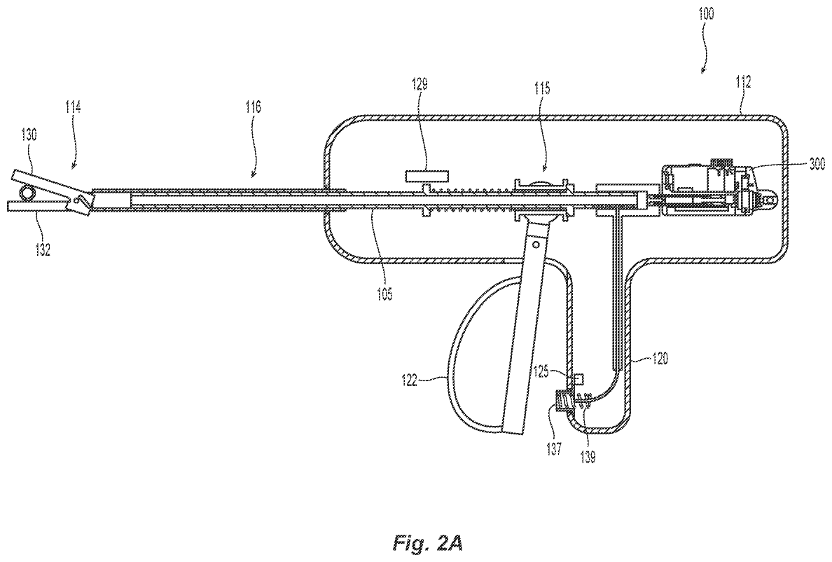

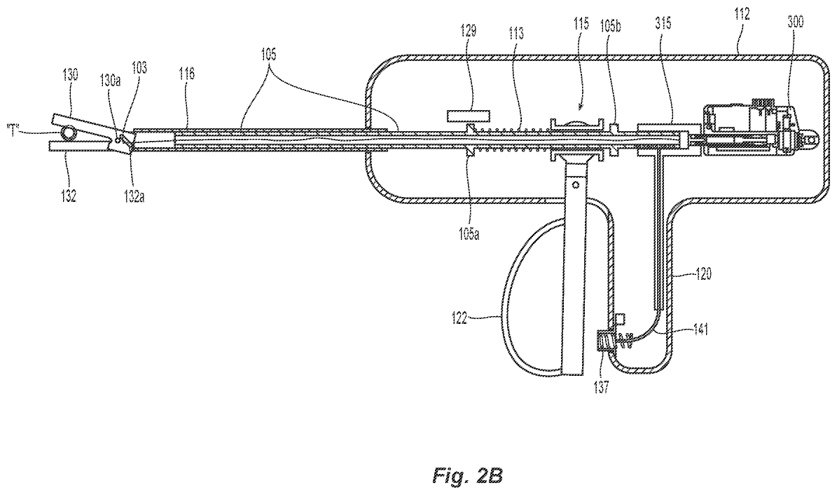

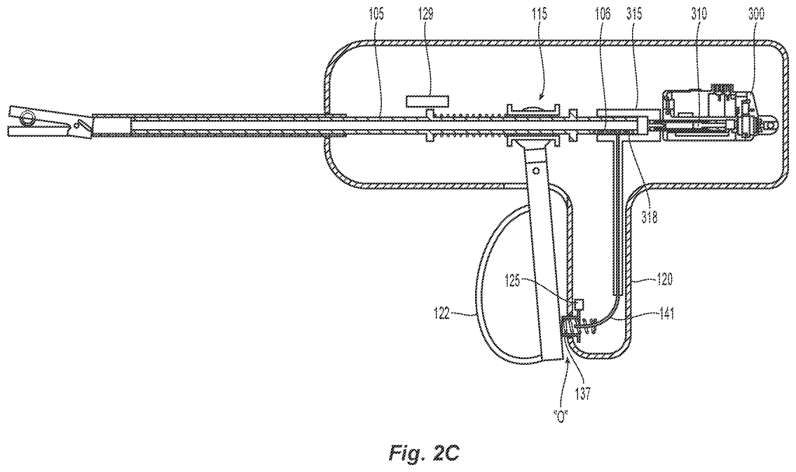

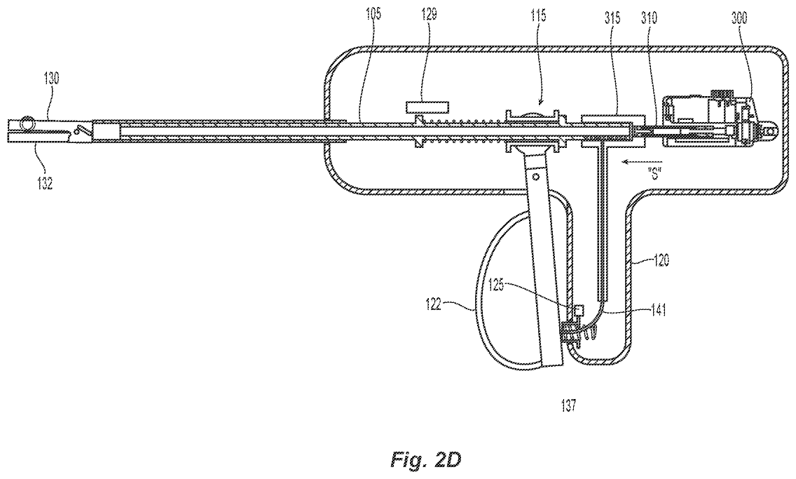

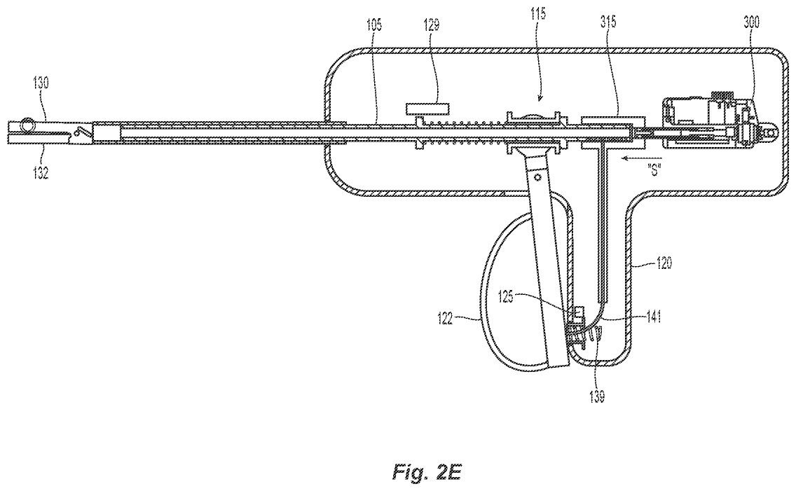

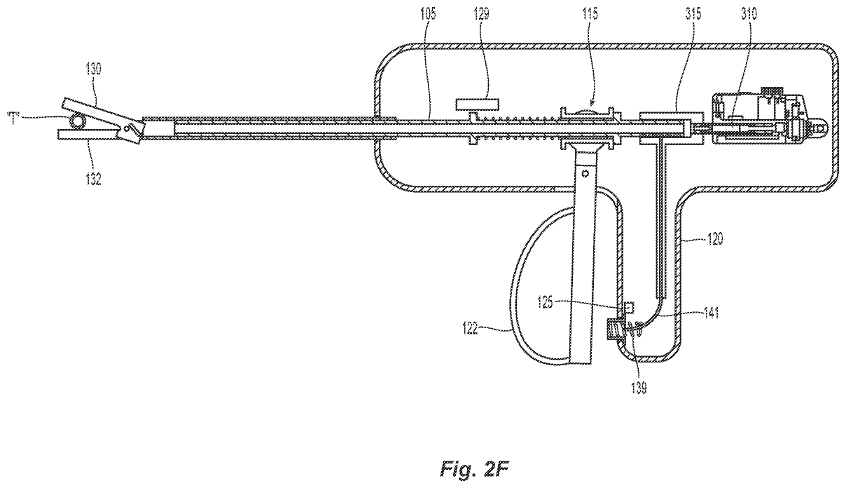

The movable handle 122 is operable to move the end effector 114 between an open configuration (FIG. 1) wherein a pair of opposed jaw members 130, 132 are disposed in spaced relation relative to one another, various degrees of grasping configurations, and to a clamping position for sealing tissue as shown in FIGS. 2A-2E. Approximation of the movable handle 122 relative to the stationary handle 120 serves to move the end effector 114 to the grasping and closed configurations and separation of the movable handle 122 from the stationary handle 120 serves to move the end effector 114 to the open configuration.

To electrically control the end effector 114, the stationary handle 120 supports a depressible button 137 thereon, which is operable by the user to both control the clamping pressure and initiate and terminate the delivery of electrosurgical energy to the end effector 114 for sealing tissue as explained in more detail below. More particularly, button 137 is engageable by a proximal side of the moveable handle 122 upon proximal movement of the moveable handle 122 to an actuated or proximal position. Button 137 includes a base ring disposed within stationary handle 120 that is configured to be slidingly received therein against the force of a spring 139 upon actuation of handle 122 when moved to the proximal position (see FIGS. 2C-2E), the purposes of which will be explained below. The button 137 is in electrical communication with a motor 300 and an electrosurgical generator “G” via suitable electrical wiring (not explicitly referenced) extending from the housing 112 through a cable 143 extending between the housing 112 and the electrosurgical generator “G”. The generator “G” may include devices such as the LigaSure® Vessel Sealing Generator and the ForceTriad® Generator sold by Covidien. Button 137 may be configured to simply control the motor 300 and a separate switch (not shown) may be utilized to control electrosurgical activation of the jaw members 130, 132 to seal tissue.

The end effector 114 may be moved from the open configuration (FIG. 1) wherein tissue “T” is received between the jaw members 130, 132, a range of grasping positions wherein the surgeon handles and manipulates the tissue “T” (FIGS. 2A-2B), and to the clamping configuration (FIGS. 2C-2E), wherein the tissue “T” is clamped under the necessary forces to seal tissue. The jaw members 130, 132 pivot about a pivot pin 144 to move the end effector 114 between positions. To provide an effective tissue seal, a pressure within a range between about 3 kg/cm² to about 16 kg/cm² and, typically, within a working range of about 7 kg/cm² to about 13 kg/cm², may be applied to the tissue. Also, in the closed configuration, a separation or gap distance is maintained between jaw members 130, 132 of about 0.001 inches to about 0.010 inches and, typically, between about 0.003 inches to about 0.006 inches, may be provided. In some embodiments, stop members (not shown) may be provided on the inner facing surfaces of the jaw members 130, 132 made of a heat-resistant ceramic in any suitable number, arrangement, and/or configuration, depending on a particular purpose.

The jaw members 130, 132 are electrically coupled to cable 143, and thus to the generator “G” (e.g., via respective suitable electrical wiring extending through the elongated shaft 116) to provide an electrical pathway to the jaw members 132, 130. Once a tissue seal is established, a knife blade (not shown) may be advanced to transect the sealed tissue.

Referring generally to FIG. 1, movable handle 122 is pivotably coupled within housing about a pivot 111 to a drive collar 115 including proximal and distal stops 117a, 117b. Drive collar 115, upon movement thereof via actuation of handle 122, translates an internally-disposed drive rod 105 within shaft 116 which, in turn, moves the jaw members 130, 132 between the open, grasping and closed positions. More particularly, a distal end of the drive rod 105 actuates a drive pin 103 within a pair of respective cam slots 130a, 132a disposed within the jaw members 130, 132 to cam the jaw members 130, 132 to the closed configuration about tissue “T”.

As mentioned above, as the surgeon actuates the handle 122, energy may be applied to seal tissue by way of in-line actuation. With typical in-line activation instruments, as the moveable handle 122 is moved to a fully actuated or proximal position, the proximal portion of handle (or an element extending therefrom) depresses the button 137, thereby activating the generator “G” to deliver electrosurgical energy to the end effector 114 to delivery energy for tissue treatment. As mentioned above, with tissue sealing, surgeons typically have to generate a high clamping force prior to initiating energy delivery which with in-line devices requires the surgeon to hold the high clamping force during the seal cycle which can cause fatigue especially over repeated seal cycles.

As shown in FIG. 1 and as mentioned above, a motor 300 is disposed within the housing 112 and may be operably coupled to generator “G” or some other internal or external power supply (not shown). Motor 300 includes shaft 310 which drives a motor coupler or gear box 315. Gear box 315, in turn, includes a series of gears 318 which are configured to operably mesh with a corresponding set of gears 106 disposed on the proximal end of drive rod 105. A key 141 extends from gear box 315 and is operably coupled to the button 137. The key 141 is configured to move the gears 318 into engagement with the gear 106 to offload the forces required to clamp the tissue “T” once the handle 122 has reached a certain position as explained in more detail below.

Referring now to FIGS. 2A-2E, as mentioned above, handle 122 is moved by the surgeon between an open position, through a range of grasping positions and to a clamping position for sealing which, in turn, moves the jaw members 130, 132, respectively, from a first position wherein jaw member 130 is spaced from tissue “T” disposed therebetween (FIG. 1), a second position wherein both jaw members are slightly touching tissue to firmly grasping tissue “T” (FIGS. 2A-2C) and to a third position wherein the tissue “T” is clamped under the appropriate pressure to generate a tissue seal (FIGS. 2D-2E) . Motor 300 offloads the clamping forces for the surgeon prior to electrical activation. In other words, the surgeon is free to manipulate and grasp tissue “T” as denoted in FIGS. 2A-2C, however, prior to generating the necessary forces required for sealing tissue, the in-line activation features of forceps 100 alleviate over exertion by the surgeon and the internal motor 300 generates the clamping force by actuating the drive rod 105.

More particularly, as mentioned above, button 137 includes a base ring 138 that is slidingly received in stationary handle 120 against the force of spring 139 such that, upon actuation of handle 122 to the proximal position, the button 137 and base ring 138 move into stationary handle 120 and against spring 139 (see FIGS. 2C-2E). Base ring 138 moves relative to a position encoder 125 disposed within stationary handle 120 which is configured to provide feedback to the motor 300 regarding the relative position of button 137 which, in turn, will instruct the motor 300 to activate and engage the drive rod 105 once the button 137 has been sufficiently depressed, i.e., the surgeon has grasped the handle 122 passed the sufficient offload position “O” (See FIG. 2C v FIG. 2E). In addition to the motor 300 activating via the instructions from the encoder 125, as the handle 122 is grasped to position “O” (FIG. 2C) and the base ring 138 moves passed encoder 125, key 141 is moved to urge gears 318 of gear box 315 into engagement with gears 106 of drive rod 105 such that the drive shaft 310 of the motor 300 can drive the drive rod 105 in the direction “S” to clamp the jaw members 130, 132 and offload or relieve the handle 122 and the surgeon from generating the higher forces required for sealing tissue (FIGS. 2D and 2E).

As can be appreciated, the surgeon is free to grasp and manipulate tissue “T” with relatively light tissue pressure between jaw members 130, 132 so as to not damage tissue during manipulation thereof and requiring relatively light handle pressure on the surgeon's hand, e.g., well below the pressure required for vessel sealing (about 3 kg/cm² to about 16 kg/cm² ). Once the surgeon selects tissue “T” to be sealed, the surgeon simply grasps the tissue “T” between the jaw members 130, 132 to the position “O” and the motor 300 is activated to clamp the tissue “T” and initiate the Ligasure® cycle as explained in detail below. The surgeon maintains control over the handle 122 and motor 300 (while the motor 300 assists in maintaining the sealing pressure) but with only minimal pressure on the handle 122 greatly reducing surgical fatigue.

In use, and as shown in detail in the various FIGS. 1-2F, the surgeon initially orients tissue “T” between jaw members 130, 132. Movement of the drive rod 105 to cam the jaw members 130, 132 to a grasping position is proportional to the relative movement of the handle 122 to the stationary handle 120 about pivot 111. Motor 300 and motor drive shaft 310 are initially disengaged from the drive rod 105 through this initial grasping range of motion until engagement of button 137 as explained above. The surgeon grasps the handle 122 relative to stationary handle 120 to manipulate tissue “T” applying gradually increasing grasping pressure onto the tissue “T” as denoted in FIGS. 2A-2C without actuating button 137. As such, the surgeon is free to grasp and manipulate tissue until a sealing site is determined (FIGS. 1-2B). Typical systems employing 1:1 ratios of handle to jaw motion are contemplated. As more grasping pressure is applied, drive collar 115 gradually separates from the proximal collar stop 105b against the bias of spring 113.

A position encoder 129 records the relative position of a distal collar stop 105a of the drive rod 105 to the encoder 129 which may be used during or after a seal cycle to assess seal quality. A force transducer 105c may also be included to sense a linear force on drive rod which may be used during a seal cycle or after to assess seal quality.

Once a tissue sealing site is determined, the surgeon grasps the handle 122 relative to stationary handle 120 and applies grasping pressure until position “O” is reached (FIG. 2C). At this point, the motor 300 offloads the pressure requirements from the handle 122 and operably couples to the drive rod 105. More particularly, at position “O” two operations are initiated simultaneously (or substantially simultaneously): first, the button 137 is actuated by the proximal facing portion of the handle 122 to urge the key 141 to, in turn, engage the gears 318 of the gear box 315 of the motor 300 into engagement with the gears 106 on the drive rod 105; and second, the encoder 125 is instructed to communicate with the motor 300 that the button 137 has been moved and the motor 300 is now engaged to the drive rod 105.

Once the encoder 125 senses that the button 137 has been moved and the motor 300 is engaged, the motor 300 initially moves drive rod 105 via rotation of drive shaft 310 and gears 318 to a fully clamped or fully grasped position as shown in FIG. 2D. At this point the tissue “T” is fully grasped and under control of the motor 300 (versus under the control of the handles 122 for manipulation purposes). It is important to note, that the surgeon still has the option to release the handle 122 from the button 137 which will disengage the motor 300 and re-assign operational control back to the handle 122 for additional grasping and manipulation if needed.

If the surgeon wishes to enable a LigaSure® seal cycle and seal the tissue “T” disposed between the jaw members 130, 132, the surgeon further actuates the handle 122 passed encoder 125 which communicates with the motor 300 to further rotate drive shaft 310 and gears 318 to move the gear box 315 and drive rod 105 distally and cam the jaw members 130, 132 under more pressure to seal tissue. Sealing pressures within the range of about 3 kg/cm² to about 16 kg/cm² are contemplated. Once the proper sealing pressure is provided between the jaw members 130, 132, a signal may be communicated to the generator “G” or the seal cycle may be automatically initiated depending upon a particular purpose. Alternatively, the surgeon may manually initialize sealing by activating a switch (not shown). The shaft position sensor 129 and the force transducer 105c may be utilized to help improve seal quality by providing various feedback controls to the motor of generator “G” during a seal cycle.

Once a seal is complete, the surgeon simply releases the handle 122 and the spring 113 returns the drive rod 105 proximally which, in turn, returns the jaw members 130, 132 to a more open orientation. The motor 300 may be configured with a reverse gear (not shown) to facilitate initial opening of the jaw members 130, 132 depending upon a particular purpose. As the surgeon begins to release the handle 122 relative to the handle 120 and as base ring 138 moves distally along encoder 125 (i.e., after a successful seal cycle), the motor 300 may begin to reverse and begin to release the tissue “T” from the jaw members 130, 132. Reversing the motor 300 will rotate the drive shaft 310 in the opposite direction which, in turn, will retract the drive rod 105 proximally (and gear box 315 proximally) and cam the jaw members 130, 132 to a more open position.

Once the button 137 is disengaged from the handle 122, the encoder 125 will instruct the motor 300 to stop the reverse gear while at the same time the key 141 will release gear 318 from gear 106 allowing the handle 122 to resume operational control of the drive rod 105 and jaw members 130, 132 and the handle 122 will reset back to a grasping and manipulation function as shown in FIGS. 1-2C.

It is important to note that during sealing, the surgeon maintains the same grasping pressure with button 137 engaged but sealing pressure is generated by the motor 300 to seal tissue. As a result, surgical fatigue is minimized during procedures with repeated seal cycles while still providing surgical safety since surgeon can at any time opt to release the jaw members 130, 132 by releasing button 137 and disengage the motor 300.

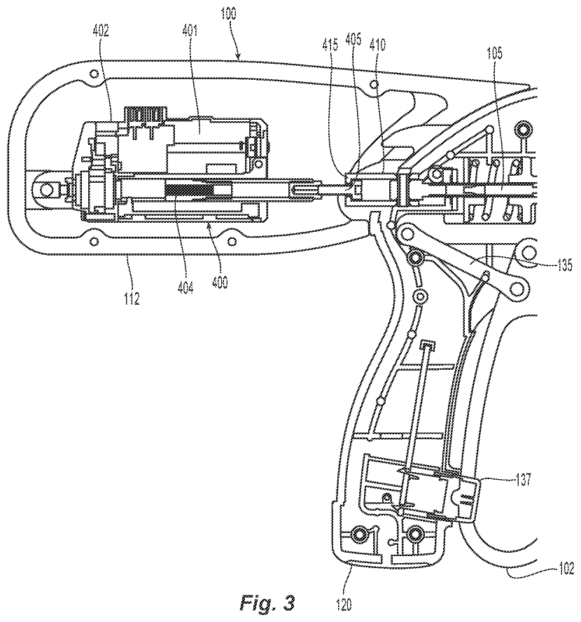

Turning to FIG. 3 which shows another embodiment of an over-ride 400 motor configured to offload the clamping or LigaSure® forces for the surgeon prior to electrical activation. Similar to the forceps 100 and override 300 described above, the surgeon is free to manipulate and grasp tissue “T” as denoted in FIGS. 2A-2C, however, prior to generating the necessary forces required for sealing tissue, the in-line activation features of forceps 100 and override 400 also alleviate over exertion by the surgeon and the internal motor 400 generates the sealing force by actuating the drive rod 105

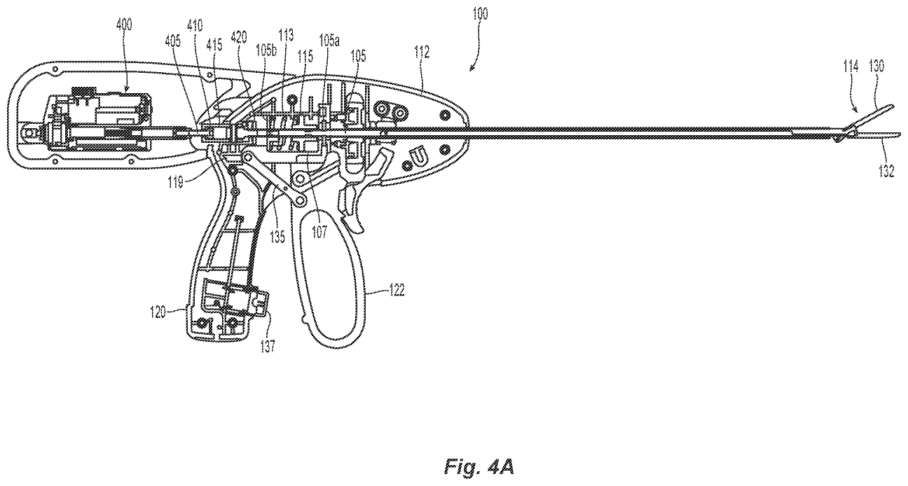

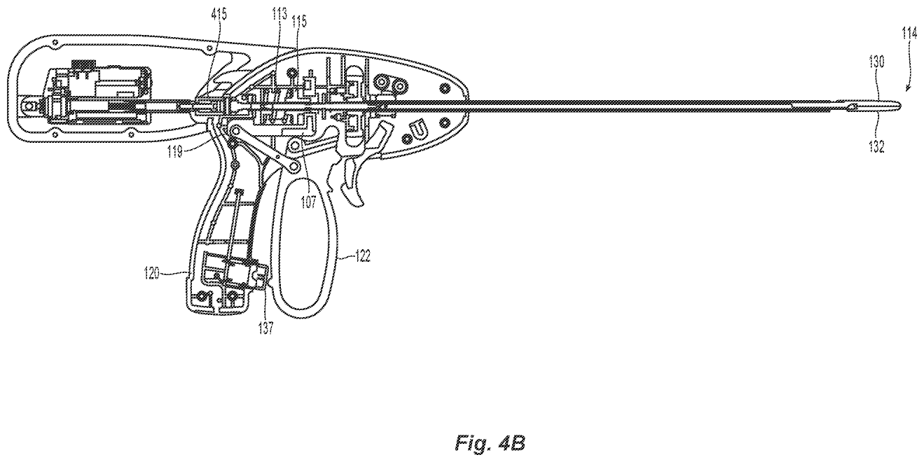

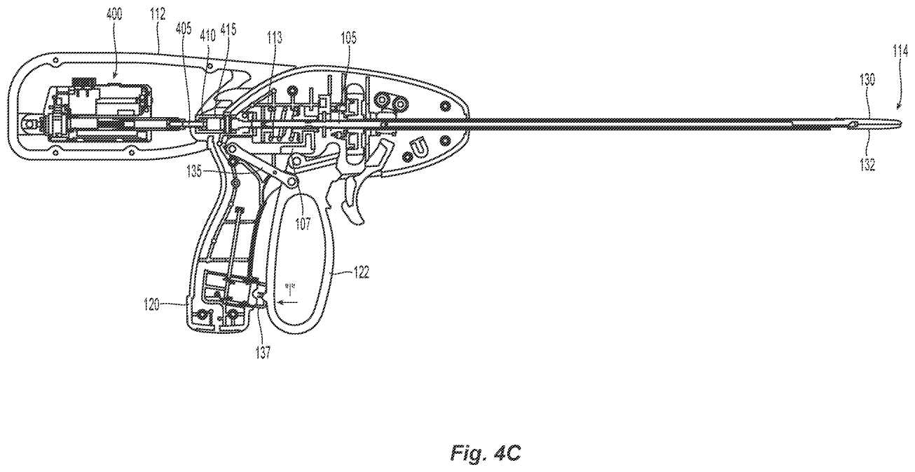

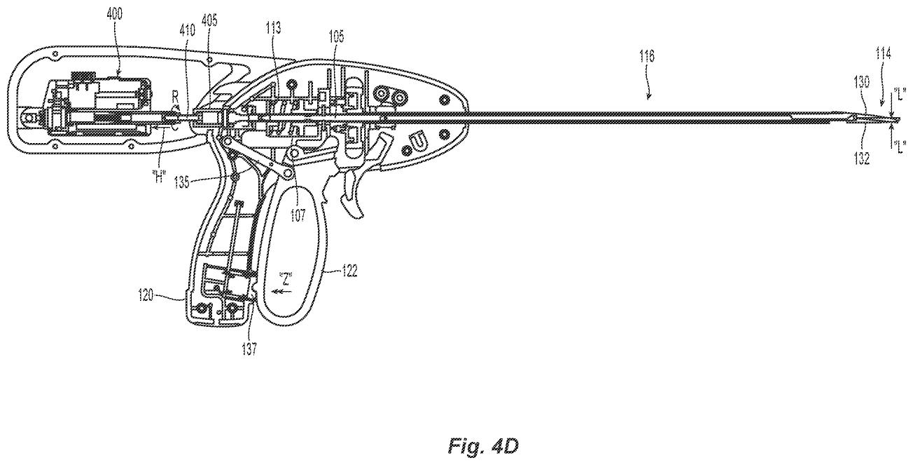

The forceps 100 of FIG. 3 is similar to the forceps 100 described above and, as such, is briefly summarized herein and only those features that are different will be described in detail. Movable handle 122 is operable to move the end effector 114 between an open and closed positions (FIGS. 4A and 4D). Handle 120 supports a two-stage button 137 which activates electrosurgical energy and controls grasping, clamping sealing pressure.

As described above, as the surgeon actuates the handle 122 to grasp, manipulate tissue and eventually seal tissue, the surgeon must overcome the spring forces of the handle necessary to generate the forces required for sealing. Once the desired tissue is disposed and clamped between the jaw members 130, 132, energy is applied to seal tissue by way of in-line actuation by further actuating the handle 122 relative to handle 120. Typically, surgeons have to generate and maintain a high clamping force prior to initiating energy delivery with in-line devices during the seal cycle which can cause fatigue especially over repeated seal cycles.

Forceps 100 of FIG. 3 shows another embodiment configured to alleviate this issue by providing override 400. Override 400 includes motor 402 encased in motor housing 401 and configured to drive a rotary shaft 404. Shaft 404, in turn, couples to distal screw 405 having a screw head 410 slidingly disposed within a proximal shaft connector 415. A seal or guide 420 controls the movement of shaft 105 into connector 415.

A toggle 135 connects a proximal portion of handle 122 with a proximal end of a drive carriage 107 which is configured to support the drive rod 105, drive collar 115, and drive spring 113 between drive stops 105a, 105b. Toggle 135 supports the sliding movement of the drive carriage 107 along a rail 119 defined in the proximal end of housing 112 during actuation of the handle 122.

Operation of the override 400 is described with reference to FIGS. 4A-4D. FIG. 4A shows the handle 122 is an unactuated position with the jaw members 130, 132 fully open for assessing and manipulating tissue. The carriage 107 in disposed in a distal-most orientation and the screw head 410 is disengaged with the proximal shaft connector 415, i.e., the motor 402 is not engaged.

FIG. 4B shows the handle 122 actuated relative to handle 120 but not engaged with button 137. As such, as handle 122 rotates, handle 122 drives spindle 115 against the force of spring 113 to move carriage 107 along rail 119 proximally under the guidance of toggle 119 pulling drive shaft 105 proximally to cam jaw member 130 relative to jaw member 132 about tissue. Spring 113, in this instance, is a low compression spring as there is no requirement to generate high compression forces for sealing tissue and, as such, surgeon fatigue is minimized over successive use. Low compression spring is defined to have a compression force in the range of about XXX N to about XXXXX N.

FIG. 4C shows the handle 122 actuated to a first stage “1” of in-line actuation (represented by a single-headed arrow) wherein electrosurgical energy is initiated and a clamping force is increased via the motor 402. More particularly, upon activation of the activation button 137, electrosurgical energy is activated along with motor 402 to rotate the rotary shaft 404 in the direction “R” which draws the screw head 410 against the proximal shaft connector 415. As such, the motor 402 overrides or disengages the handle 122 and pulls the drive shaft 105 proximally generating additional closing force to cam the jaw members 130, 132 against the tissue. As can be appreciated this alleviates the surgeon from generating the forces for sealing tissue.

FIG. 4D shows the handle 122 actuated to a second stage “2” of in-line actuation (represented by a double-headed arrow) wherein electrosurgical energy is still initiated via activation of button 137 and a higher force “H” is generated by the motor 402 by further rotating the rotary shaft 404 in the direction “R” which draws the screw head 410 against the proximal shaft connector 415. This, in turn, pulls the drive shaft 105 proximally generating a LigaSure® sealing force “L” between the jaw members 130, 132 to seal tissue therebetween. Again, the motor 402 overrides or disengages the handle 122 and pulls the drive shaft 105 proximally generating the necessary forces to seal tissue between the jaw members 130, 132. Once again, this alleviates the surgeon from generating the forces for sealing tissue.

Once tissue is sealed and in a similar manner as described above with reference to FIGS. 2A-2D, when the surgeon releases the handle 122 the override 400 and motor 402 are deactivated and the handle 122 resumes control of the jaw members 130, 132 to release the tissue.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

1. A surgical forceps for sealing tissue, comprising:
a housing including an elongated drive rod having an end effector assembly disposed at a distal end thereof, the end effector including first and second jaw members configured to seal tissue upon electrical activation thereof;
a stationary handle depending from the housing and configured to support a clamping override actuator disposed therein, the clamping override actuator operably coupled to a motor; and
a moveable handle operably coupled to the drive rod and movable relative to the stationary handle through an initial range of motion to move the drive rod upon actuation thereof without contacting the clamping override actuator to pivot at least one of the first or second jaw members relative to the other of the first or second jaw members to grasp tissue therebetween, the moveable handle configured to contact the clamping override actuator through a further range of motion to actuate the clamping override actuator to engage the motor with the drive rod to further move the drive rod to generate a higher clamping force to seal tissue,
wherein the clamping override actuator is disposed in the actuation path of the moveable handle.

2. The surgical forceps for sealing tissue according to claim 1, wherein the clamping override actuator includes an encoder configured to communicate to the motor that the clamping override actuator has been contacted by the moveable handle, which, in turn, recognizes that the motor is mechanically engaged with the drive rod.

3. The surgical forceps for sealing tissue according to claim 1, wherein the clamping override actuator includes a key operably connected thereto that is movable therewith, the key configured to urge at least one gear from the motor into engagement with at least one corresponding gear on the drive rod.

4. The surgical forceps for sealing tissue according to claim 3, wherein the motor includes a gear box disposed on a drive shaft that is configured to matingly engage a corresponding series of gears disposed on the drive rod.

5. The surgical forceps for sealing tissue according to claim 1, wherein the clamping override actuator is biased against a spring disposed within the stationary handle.

6. The surgical forceps for sealing tissue according to claim 1, wherein the moveable handle is moveable about a pivot through the initial range of motion to force a drive collar against the drive rod to pivot the jaw members relative to one another for grasping tissue.

7. The surgical forceps for sealing tissue according to claim 6, wherein the drive collar compresses a spring against a drive collar stop to regulate the grasping pressure between the jaw members.

8. The surgical forceps for sealing tissue according to claim 7, further comprising a position encoder disposed proximate the drive collar stop and configured to report the position of the drive collar stop relative thereto during sealing.

9. The surgical forceps for sealing tissue according to claim 7, further comprising a force transducer disposed on a portion of the drive rod proximate the drive collar stop and configured to sense a linear force on the drive rod during sealing.

10. A surgical forceps for sealing tissue, comprising:
a housing including an elongated drive rod having an end effector assembly disposed at a distal end thereof, the end effector including first and second jaw members configured to seal tissue upon electrical activation thereof;
a stationary handle depending from the housing and configured to support a clamping override actuator disposed therein, the clamping override actuator operably coupled to a motor; and
a moveable handle operably coupled to the drive rod and movable relative to the stationary handle through an initial range of motion which moves the drive rod upon actuation thereof to pivot at least one of the first or second jaw members relative to the other of the first or second jaw members to grasp tissue therebetween under a first clamping pressure by pivoting a drive collar against a drive stop, the moveable handle configured to transition through a further range of motion to an override position to actuate a clamping override actuator to engage a motor with the drive rod to further move the drive rod to generate a higher clamping force to seal tissue,
wherein the clamping override actuator is disposed in the actuation path of the moveable handle.

11. The surgical forceps for sealing tissue according to claim 10, wherein the clamping override actuator includes an encoder configured to communicate to the motor that the clamping override actuator has been contacted by the moveable handle, which, in turn, recognizes that the motor is mechanically engaged with the drive rod.

12. The surgical forceps for sealing tissue according to claim 10, wherein the clamping override actuator includes a key operably connected thereto that is movable therewith, the key configured to urge at least one gear from the motor into engagement with at least one corresponding gear on the drive rod.

13. The surgical forceps for sealing tissue according to claim 12, wherein the motor includes a gear box disposed on a drive shaft that is configured to matingly engage a corresponding series of gears disposed on the drive rod.

14. The surgical forceps for sealing tissue according to claim 10, wherein the clamping override actuator is biased against a spring disposed within the stationary handle.

15. The surgical forceps for sealing tissue according to claim 10, wherein the moveable handle is moveable about a pivot though the initial range of motion to force a drive collar against the drive rod to pivot the jaw members relative to one another for grasping tissue.

16. The surgical forceps for sealing tissue according to claim 15, wherein the drive collar compresses a spring against a drive collar stop to regulate the grasping pressure between the jaw members.

17. The surgical forceps for sealing tissue according to claim 10, further comprising a position encoder disposed proximate the drive collar stop and configured to report the position of the drive collar stop relative thereto during sealing.

18. The surgical forceps for sealing tissue according to claim 10, further comprising a force transducer disposed on a portion of the drive rod proximate the drive collar stop and configured to sense a linear force on the drive rod during sealing.

19. A method of sealing tissue with a motorized assist, comprising:
orienting tissue between first and second jaw members of an end effector assembly supported at a distal end of an elongated drive rod extending from a housing;
moving a movable handle through an initial range of motion relative to a stationary handle depending from the housing to move the drive rod to approximate the jaw members to grasp tissue disposed therebetween under a first pressure;
moving the handle through a further range of motion to transition to an override position of the moveable handle so as actuate a clamping override actuator which engages a motor with the drive rod, the motor configured to relieve the moveable handle from the drive rod and further move the drive rod to generate higher clamping forces for sealing tissue; and
releasing the clamping override actuator upon seal completion to disengage the motor and re-engage the moveable handle with the drive rod to open the jaw members upon movement thereof to release the tissue.

20. (canceled)
